(19) United States
(12) Patent Application Publication
(10) Pub. No.: US 2015/0122262 A1
(43) Pub. Date: May 7, 2015

(54) PATIENT INTERFACE DEVICE HAVING TIGHTENING INDICATOR

(71) Applicant: KONINKLIJKE PHILIPS N.V., EINDHOVEN (NL)

(72) Inventor: Harmina Christina Zeijlstra, Breda (NL)

(21) Appl. No.: 14/363,511

(22) PCT Filed: Dec. 6, 2012
(86) PCT No.: PCT/IB2012/057023

§ 371 Date: Jun. 6, 2014

Related U.S. Application Data
(60) Provisional application No. 61/569,311, filed on Dec. 12, 2011.

(51) Int. Cl. A61M 16/06; A61M 16/00

(52) U.S. Cl. CPC A61M 16/0683; A61M 16/0057; A61M 16/0616; A61M 16/0644; A61M 2205/582; A61M 2205/3344; A61M 2205/581; A61M 2205/584; A61M 2205/44
USPC 128/205.25

(57) ABSTRACT

The present invention relates to patient interface, comprising a patient interface (12; 112) for delivering a gas to a user arranged to be tightened towards the user's face, and at least one tightening indicator (48; 148) for indicating an optimum tightening position of the patient interface (12; 112), wherein the at least one tightening indicator (48; 148) is arranged at the patient interface (12; 112) and configured to indicate the optimum 5 tightening position at a predetermined position of the patient interface with respect to the user's face.

### FIELD OF THE INVENTION

The present invention relates to a patient interface device (often also called respiratory interface device) adapted to deliver a gas flow to a user. Further, the present invention relates to a patient interface system comprising a patient interface device and tightening means.

### BACKGROUND OF THE INVENTION

There are numerous situations where it is necessary or desirable to deliver a gas flow non-invasively to the airway of a user. Typical examples are treatments of medical disorders, such as sleep apnea syndrome, in particular, obstructive sleep apnea (OSA), or congestive heart failure.

Non-invasive ventilation and pressure support therapies involve the placement of a patient interface device for a patient or a user in general, which typically comprises a patient interface (also called facial mask), such as a nasal mask that covers the nose, a nasal oral mask that covers the nose and the mouth, or a full face mask that covers the patient's face. Typically a headgear is used to maintain such a facial mask on the face of a user wherein the headgear can be tightened to maintain the facial mask in a preferable position. Since such masks are typically worn for an extended period of time, it is important for the headgear to maintain the mask in a tight position on the one hand and avoid typical patient's discomfort which can be related to red marks or other complaints within the user's face. It shall be noted that if the term “mask” is used herein, this shall not be understood as a limitation to this special embodiment rather than referring to patient interfaces as described herein in general, where applicable and if not stated otherwise.

Such a headgear for a facial mask comprising an apparatus for indicating the strapping force is disclosed in WO 2011/048518 A1. The apparatus includes a first portion configured to be selectively connectable to a connecting element of the facial mask of the patient interface device, a second portion configured to be selectively connectable to a strap of the headgear, and a force indicating mechanism coupled to the first portion and the second portion. The force indicating mechanism is configured to indicate a force being applied by the headgear in response to the second strap portion being pulled by the strap of the headgear.

US 2003/0144596 A1 discloses a strap structure arranged to fasten and position a sensing device. The strap comprises an elongation indicating means for indicating the tightening of the elastic strap and the sensing device.

One major drawback of the known indicating devices is that they only indicate the tension of the straps but insufficiently indicate the pressure of the mask onto the face which is crucial to guarantee a sealed fitting of the mask within the face on the one hand and to avoid an undesired over-tightening of the facial mask onto the face which typically causes discomfort like facial red marks, pressure source and/or open wounds.

### SUMMARY OF THE INVENTION

It is an object of the present invention to provide a patient interface device and a corresponding patient interface system which are easy to handle and which help the user to avoid an undesired over-tightening of the patient interface device on the one hand and to ensure a sealed fitting of the patient interface device on the other hand.

According to a first aspect of the present invention, a patient interface device is presented that comprises a patient interface for delivering a gas (e.g. air, air enriched in oxygen, a mixture comprising oxygen and a medicine) to a user arranged to be tightened towards the user's face, and at least one tightening indicator for indicating an optimum tightening position of the patient interface, wherein the at least one tightening indicator is arranged at the patient interface and configured to indicate the optimum tightening position at a predetermined position of the patient interface with respect to the user's face.

According to another aspect of the present invention, a patient interface system is presented that comprises a patient interface device according to the first aspect of the present invention and a tightening means being connected to the patient interface device enabling the user to tighten the patient interface device towards the user's face.

Preferred embodiments of the invention are defined in the dependent claims.

The present invention is based on the idea to provide a patient interface device comprising at least a patient interface and a tightening indicator wherein the tightening indicator is adapted to indicate an optimum tightening position of the patient interface device onto the face of a user. An optimum tightening position is reached, when the patient interface is in a position where it obtains a good sealing between the mask and the user's face on the one hand and the tightening means is not over-tightened so that the mask does not cause undesired discomfort which might lead to facial red marks, pressure source and/or open wounds.

According to the present invention, the indication of the optimum tightening position of the patient interface within the user's face is accomplished by use of the tightening indicator indicating the optimum tightening position at a predetermined position of the patient interface with respect to the user's face. The tightening indicator therefore provides a simple means for directly giving a feedback to the user when the optimum tightening position of the patient interface is reached. This tightening indicator may previously be adjusted to the individual user since the optimum tightening position is generally different for each user and each patient interface.

There are different embodiments for this indication. In one embodiment the at least one tightening indicator is arranged at a distance from the user's face in a non-tightened state of the patient interface and configured to get closer to the user's face upon tightening the patient interface and to contact the user's face indicating the optimum tightening position. Thus, when the tightening indicator contacts the user's face this is interpreted as an indicating that the patient interface is correctly tightened. This physical contact may be directly realized (i.e. felt) by the user, or the contact may cause the indicator to issue any kind of signal, e.g. an audible, visual or sensible signal.

In another embodiment the at least one tightening indicator is configured to indicate the optimum tightening position when reaching a predetermined pressure of the patient interface onto the user's face. The predetermined pressure, which is preferably determined in advance when adjusting the patient interface to the user, may be reached when the tightening indicator contacts the user face's (i.e. the predetermined pressure is rather low and is exceeded already when the indicator slightly touches the user's skin) or may be a higher predetermined pressure when the patient interface is pressed against the user's face with increasing pressure. In such embodiments the tightening indicator may generally be any kind of chemical or physical indicator that is able to provide, in response to a predetermined pressure, any kind of signal that is perceptible by the user.

The tightening means may generally be any kind of means that allow tightening of the patient interface to the user's face. In many embodiments such tightening means comprise a headgear, e.g. including adjustable straps, which headgear may be fixedly connected to the patient interface or which may be removable or replaceable by other means. In other embodiments the tightening means may comprise guide rails or adjustable straps that are connected or connectable to the user's head in any arbitrary way.

In a preferred embodiment of the invention, the at least one tightening indicator is arranged at a top position of the patient interface for indicating the optimum tightening position at the user's forehead. This ensures that at least an optimum tightening position in the vicinity of the user's forehead can be indicated since the forehead region within a user's face is one of the regions which is typically affected by an over-tightening of the patient interface.

In a further embodiment of the invention, the at least one tightening indicator is arranged at a lower side position of the patient interface for indicating the optimum tightening position at the user's cheek. This ensures that the optimum tightening position can be indicated at least in the vicinity of the user's cheek which is advantageous since this region is one of the regions being most affected by an over-tightening of the patient interface which typically causes red marks within this region.

In a further preferred embodiment of the invention, the at least one tightening indicator is rotatably arranged for turning the tightening indicator away from the face after reaching the optimum tightening position. This embodiment is advantageous since the tightening indicator can be rotated away from the face of the user after a positioning of the patient interface which reduces the danger of an injury caused by the tightening indicator within the user's face.

In a further preferred embodiment of the invention, a plurality of tightening indicators are arranged at different positions of the patient interface for indicating the optimum tightening position at different positions of the user's face. This embodiment is advantageous since the optimum tightening position of the mask can be determined by using a plurality of tightening indicators which are preferably distributed uniformly over different positions of the patient interface which ensures a uniform compression of the patient interface within the user's face.

In a further preferred embodiment of the invention, the at least one tightening indicator is connectable to at least a plurality of positions of the patient interface. This embodiment is advantageous since only one or just a few tightening indicators can be used to indicate the optimum tightening position of a plurality of positions which are preferably distributed uniformly over the patient interface.

In a further preferred embodiment of the invention, the at least one tightening indicator is removable from the patient interface. This embodiment is advantageous since after completely removing the tightening indicator it is impossible that the tightening indicator might injure the skin of the user. The danger of damaging the tightening indicator is also reduced.

In a further preferred embodiment of the invention, the at least one tightening indicator is a signal indicator for indicating the optimum tightening position by generating a signal induced by a face contact. This embodiment is advantageous since a signal indicator generating a signal induced by a face contact, which can be for instance a sound signal or a light signal, is typically robust in use and cost-inexpensive in production. Additionally, the emitted signal eases the handling of the patient interface device, since the emitted signal gives the hint to the user when the tightening of the patient interface onto the face should be stopped.

In a further preferred embodiment of the invention, the at least one tightening indicator is a stick-like tightening element. This embodiment of the invention is advantageous since a stick-like tightening element, e.g. a rod, bar, stick or similar element, which is e.g. made from plastic material, is very cost-inexpensive and robust in use and additionally easy to clean after several using cycles.

In a further preferred embodiment of the invention, the at least one tightening indicator is a chemical indicator for indicating the optimum tightening position by generating a chemically-induced signal caused by a face contact. This embodiment is advantageous, since an induced signal which is generated by a face contact helps the user to identify the optimum tightening position of the patient interface. Such a signal can be for instance a chemically-induced color- or light signal.

In a further preferred embodiment of the invention, the patient interface comprises a first part and a second part, wherein the first part covers the mouth and/or the nose of the user and the second part is arranged at the user's forehead. This provides the advantage that the first part of the patient interface additionally helps to distribute the compression caused by the patient interface within the face of the user. In addition, this further mounting point makes it easier for the user to bring the patient interface into an optimum position within the user's face.

In a further preferred embodiment of the invention the at least one tightening indicator is arranged at the first part. This is advantageous, since the optimum tightening position can at least be indicated at the user's forehead which at least reduces pressure marks within the upper forehead region being in contact with the patient interface.

### BRIEF DESCRIPTION OF THE DRAWINGS

These and other aspects of the invention will be apparent from and elucidated with reference to the embodiment(s) described hereinafter. In the following drawings

FIG. 1 shows a patient interface device according to an embodiment of the present invention;

FIG. 2 is a side view of the patient interface device shown in FIG. 1;

FIG. 3 is a perspective view of the patient interface device of FIG. 1 in a first application;

FIG. 4 is a perspective view of a further embodiment of a patient interface device according to the present invention in a further application.

### DETAILED DESCRIPTION OF THE INVENTION

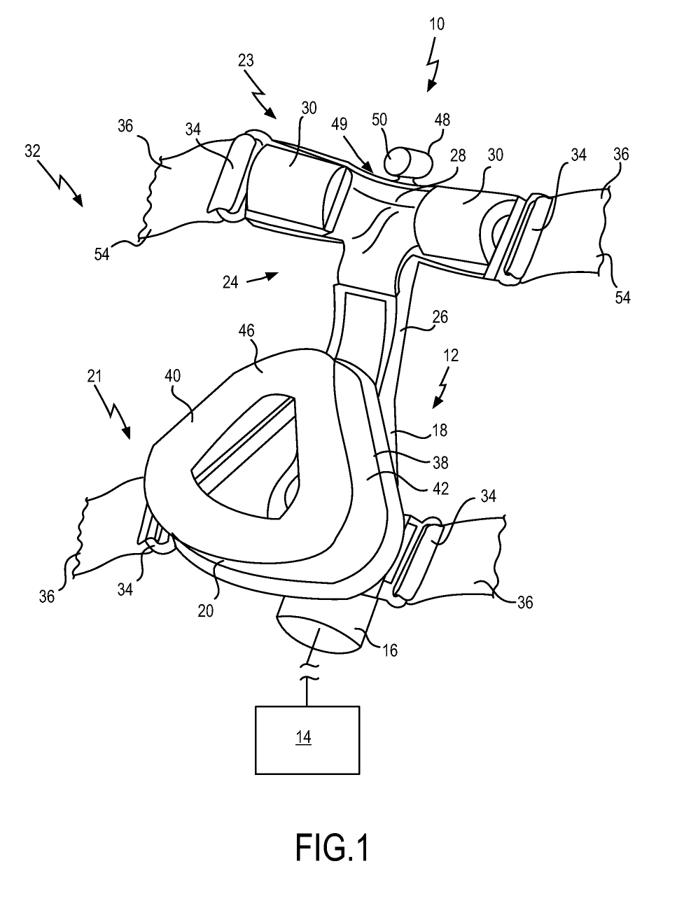

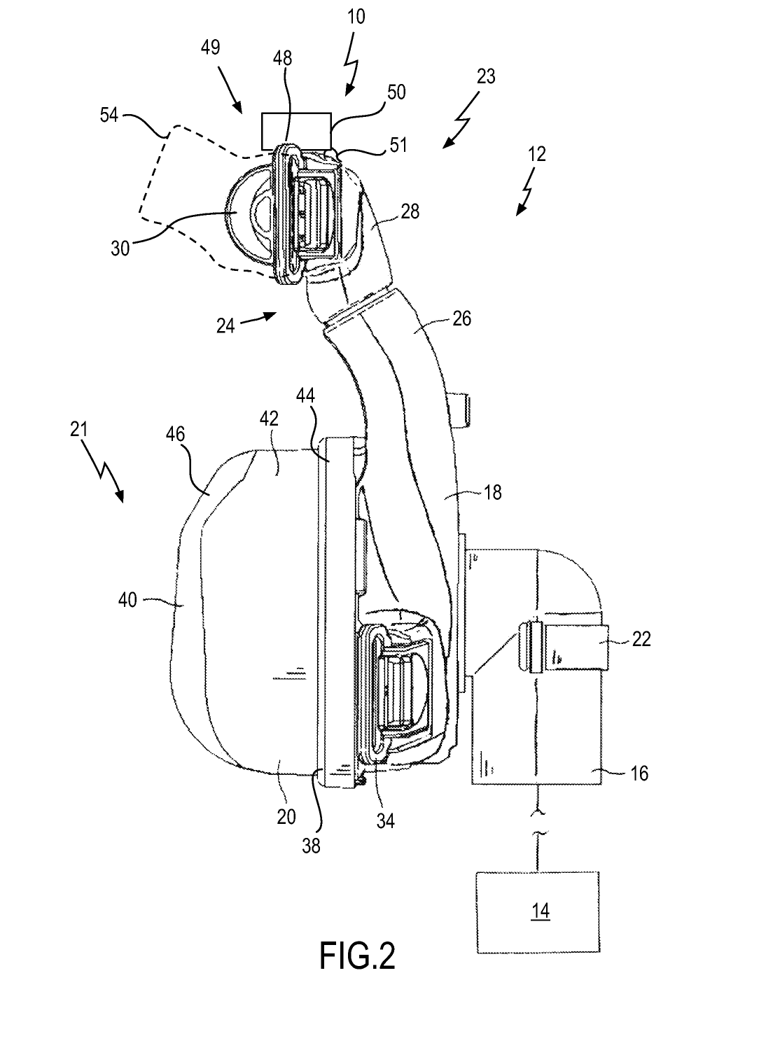

FIGS. 1 and 2 illustrate an exemplary embodiment of a patient interface device 10 according to the present invention. The patient interface device 10 comprises a patient interface 12 (hereinafter called facial mask) which is attached to a pressure generator system 14 via a patient circuit 16, as is conventionally known in the art. The facial mask 12 includes a shell 18 and a seal 20 being attached to the shell. Preferably, the patient circuit 16 is connected to the mask shell 18 so as to pivot or rotate relative to the masks shell 18 and may or may not be detachable therefrom. In short, any conventional coupling technique for joining the patient circuit 16 to the mask shell 18 is contemplated by the present invention.

The pressure generating system 14 is any device capable of generating a flow of gas or providing gas to an elevated pressure. In the illustrated exemplary embodiment shown in FIG. 2, an exhaust vent 22 is provided in the patient circuit 16 for exhausting a flow of gas to the ambient atmosphere. The used exhaust vent 22 can be any conventional exhaust vent.

The patient interface device 10, including the facial mask 12, the shell 18 and the seal 20, can have any one of an indefinite number of configurations, shapes, and sizes. In this embodiment, the patient interface device 10 comprises a first part 21 comprising the facial mask 12 and its components and a second part 23 comprising a forehead support 24. The forehead support 24 is generally T-shaped and includes a support arm 26, which is slidably connected to a forehead support bracket 28. The forehead support bracket 28 includes a forehead pad 30 disposed on the patient's contacting side to engage the forehead of the user. It is to be understood that the present invention contemplates that the forehead support assembly 24, and its individual components, can have any one of a variety of configurations. In other examples of the invention, it is also contemplated that the forehead support 24 can be eliminated entirely.

In the illustrated embodiment, a headgear 32 (representing tightening means) attaches to the facial mask 12 via headgear clips 34. The headgear clips 34 attached to headgear straps 36, for example by inserting the headgear straps 36 into slots provided on the clips. The headgear clips 34 are selectively attachable to the facial mask shell 18 in any conventional manner. In this embodiment, the headgear clips 34 attached to each side of the forehead support bracket 28 and to each side of the lower portion of the facial mask shell 18.

It shall be noted here that other tightening means may be provided instead of a headgear 32 for tightening the facial mask, e.g. adjustable guide rails. Further, the tightening means may be removable or replaceable by other tightening means. In the context of the present application a “patient interface device” is understood as a facial mask without any tightening means, whereas a “patient interface system” shall be understood as a patient interface device including tightening means.

The seal 20 includes a first end portion 38 that couples to the mask shell 18. The first end portion 38 is generally triangular-shaped and attaches to a similarly shaped counterpart of the mask shell 18. The seal 20 includes a second end portion 40 for sealing engagement with a face of a user. A side wall 42 extends between the first end portion 38 and the second end portion 40. The seal 20 is preferably a unitary structure that attaches to a mounting portion 44 of the mask shell 18 and provides a surface at the second end portion 40 that contacts the face of the patient. The second end portion 40 of the seal 20 typically comprises a soft material forming an interface 46 ensuring a good sealant of the interface 46 between the second end portion 40 and the patient's face on the one hand and a comfortable contact of the interface 46 within the user's face on the other hand.

A patient interface device of this kind is, for instance, described in U.S. Pat. No. 7,640,933 B1, the description of which is herein incorporated by reference.

For finding the optimum position of the facial mask 12 within the user's face and an optimum tightening position of the headgear straps 36 to maintain the facial mask 12 in an optimum using position, the embodiment of the proposed patient interface device illustrated in FIGS. 1 and 2 further comprises a first tightening indicator 48. This tightening indicator 48 is arranged at a top position 49 of the facial mask 12 precisely at the forehead support bracket 28 and is, in this embodiment, located between the two headgear clips 34 of the forehead support bracket 28 for indicating an optimum tightening position of the facial mask within the user's face. The tightening indicator 48 has preferably the form of a small stick 50. In other examples, the tightening indicator 48 can be arranged at other positions of the forehead support 24 of the facial mask 12 and can have various forms and sizes. The tightening indicator 48 has an articulating connection in the form of a joint 51 with the forehead support bracket 28. In another example, the tightening indicator 48 is detachable from the forehead support bracket 28 or fixed without a joint.

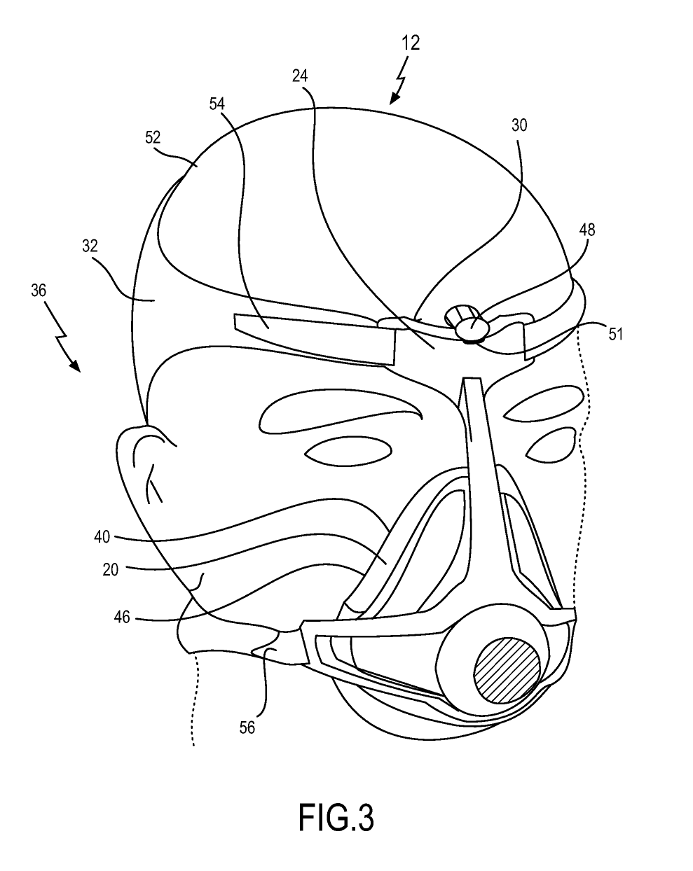

FIG. 3 illustrates positioning of the facial mask 12 and the use of the tightening indicator 48 shown in FIGS. 1 and 2. The positioning of the facial mask 12 within the user's face comprises several steps finally ending with an optimum position of the facial mask 12 within the user's face. First, the seal part 20 of the facial mask 12 is brought into a position where the seal 20 entirely covers the mouth and/or the nose of a patient. This positioning is simplified by the use of the forehead support 24 which is located at the forehead of the patient and which is in contact with the patient's forehead by the forehead pads 30. After the positioning of the facial mask 12 onto the face of the user, the headgear 32 is positioned at the back of the head 52 of the user. It is to be understood that the present invention also contemplates that the mounting of the headgear 32 can be accomplished at other positions of the user's head.

To bring the facial mask 12 into an optimum tightening position, the upper headgear strap 54 is tightened until the tightening indicator 48 is in contact with the user's forehead. By tightening headgear straps 36 in general, the second end portion 40 of the seal 20 and the forehead pads 30 are pressed onto the user's face until the second end portion 40 of the seal 20 ensures a sealed interface 46 between the second end portion 40 and the user's face, where no gas can flow out. Therefore, the straps 36 of the headgear 32 should be as loose as possible to ensure a secured sealing of the second end portion 40 within the user's face on the one hand and to avoid red marks within the user's face caused by the second end portion 40 and/or the forehead pads by an over-tightening of the headgear straps 36 on the other hand. Therefore, at least the upper headgear strap 54 is tightened until the tightening indicator 48 contacts the user's forehead. At this moment, the optimum tightening position of the facial mask within the user's face is reached. After reaching the optimum position within the use's face, the tightening indicator 48 can be detached from the user's forehead.

It is to be understood that the tightening indicator 48 can be detached from the user's forehead by articulating the tightening indicator 48 by using the joint 51 which is preferably located at the forehead support 24 of the facial mask 12. This joint 51 is preferably implemented in form of a solid body joint. It is to be understood that the present invention also contemplates a complete detachment of the tightening indicator 48 from the user's forehead and the facial mask 12.

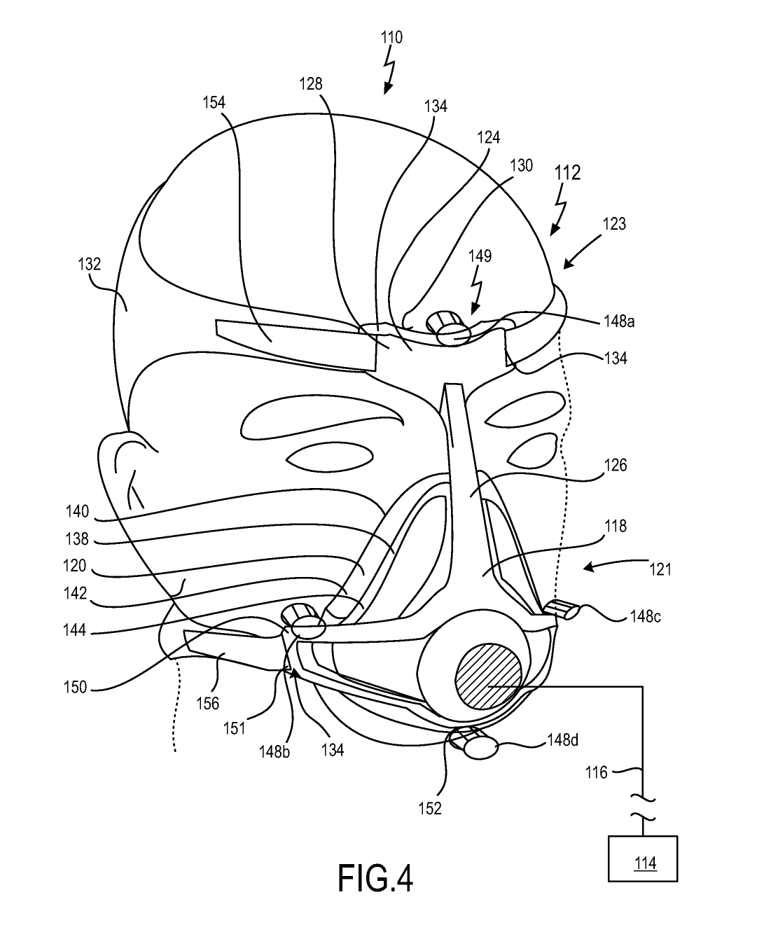

FIG. 4 shows a further embodiment of a patient interface device 110 comprising a facial mask 112. The facial mask 112 is attached to a pressure generating system 114 via a patient circuit 116. The facial mask comprises a shell body 118 and a seal 120 which comprises an interface between the patient's face and the facial mask 112. The facial mask 112 and its components form a first part 121. Additionally, a second part 123 in form of a forehead support 124 is connected to the facial mask 112 via a support arm 126 and comprises a support bracket 128 being located at the forehead of the user and comprises forehead pads 130 for smoothly contacting the forehead support 124 to the user's forehead. The facial mask 112 is maintained in position by a headgear 132 which is connected to the facial mask 112 at a plurality of headgear clips 134 via headgear straps 136. The seal 120 is connected to the facial mask 112 at a first end portion 138. A second end portion 140 of the seal 120 is in contact with the user's face. Between the first end portion 138 and the second portion 140 a sidewall 142 is located and the seal 120 is mounted at the facial mask 112 at a mounting portion 144.

In this embodiment the facial mask comprises a plurality of tightening indicators 148 wherein one tightening indicator 148a is located at a top position 149 of the facial mask 112 exactly at the forehead support 124 between the two headgear clips 134 of the forehead support bracket 128. Two further tightening indicators 148b, 148c are located at the mask shell 120 at a cheek position 150 being located in close vicinity to lower headgear clips 151 of the facial mask 112. A further tightening indicator 148d is located at a chin position 152 of the facial mask 112. The tightening indicator 148a being located at the user's forehead is arranged to indicate an optimum tightening position of an upper headgear strap 154. The lower tightening indicators 148b, 148c, 148d, being arranged at the chin position 152 of the user and the cheek position 150, indicate an optimum tightening position of a lower headgear strap 156.

To find an optimum tightening position of the entire facial mask 112, the upper headgear strap 136 is tightened until the tightening indicator 148a contacts the user's forehead and the lower headgear strap 156 is tightened until the tightening indicators 148b, 148c, 148d contact the user's face. It is to be understood that the present invention contemplates that at least one of the tightening indicators 148b, 148c, 148d should be in contact with the user' face. In the illustrated embodiment of the invention, the tightening indicators 148a, 148b, 148c, 148d are detachable and can be replaced by one another. The tightening indicators 148a, 148b, 148c, 148d can also be completely detached after finding the optimum position of the facial mask 112 within the user's face.

The tightening indicators 148a, 148b, 148c, 148d are preferably equally embodied and have an equal shape and size. In other examples, the tightening indicators 148a, 148b, 148c, 148d can also have different shapes and sizes and also tightening indicators 148 generating a signal in response to reading the tightening position are contemplated. The signal can be generated physically or chemically. The signal may be any signal that is perceptible by the user, e.g. an audible, visual or sensible signal. For instance, the tightening indicator may change its color upon reaching a predetermined pressure thus indicating that the optimum tightening position has been reached.

In other embodiments the optimum tightening position is indicated by a tightening indicator when a predetermined pressure of the facial mask onto the user's face is reached. In other words, the tightening means is not arranged and designed to indicate the optimum tightening position when it reaches contact with the user's face, but the contact may be reached earlier (e.g. already when the facial mask is put on by the user) and only when the predetermined pressure is exceeded the reaching of the optimum tightening position is indicated.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A patient interface device, comprising:
a patient interface (12; 112) for delivering a gas to a user arranged to be tightened towards the user's face, and
at least one tightening indicator (48; 148) for indicating an optimum tightening position of the patient interface (12; 112),
wherein the at least one tightening indicator (48; 148) is arranged at the patient interface (12; 112) and configured to indicate the optimum tightening position at a predetermined position of the patient interface with respect to the user's face, wherein the at least one tightening indicator is rotatably arranged for turning the tightening indicator away from the face after reaching the optimum tightening position.

2. The patient interface device of claim 1, wherein the at least one tightening indicator (48; 148) is arranged at a distance from the user's face in a non-tightened state of the patient interface (12; 112) and configured to get closer to the user's face upon tightening the patient interface (12; 112) and to contact the user's face indicating the optimum tightening position.

3. The patient interface device of claim 1, wherein the at least one tightening indicator (48; 148) is configured to indicate the optimum tightening position when reaching a predetermined pressure of the patient interface (12; 112) onto the user's face.

4. The patient interface device of claim 1, wherein the at least one tightening indicator (48; 148) is arranged at a top position (49) of the patient interface (12; 112) for indicating the optimum tightening position at the user's forehead.

5. The patient interface device of claim 1, wherein the at least one tightening indicator (48; 148) is arranged at a lower side position (152) of the patient interface (12; 112) for indicating the optimum tightening position at the user's cheek.

6. (canceled)

7. The patient interface device of claim 1, wherein a plurality of tightening indicators (48; 148) are arranged at different positions of the patient interface (12; 112) for indicating the optimum tightening position at different positions of the user's face.

8. The patient interface device of claim 1, wherein the at least one tightening indicator (48; 148) is connectable to at least a plurality of positions of the patient interface (12; 112).

9. The patient interface device of claim 1, wherein the at least one tightening indicator (48; 148) is removable from the patient interface (12; 112).

10. The patient interface device of claim 1, wherein the at least one tightening indicator (48; 148) is a signal indicator for indicating the optimum tightening position by generating a signal caused by a face contact.

11. The patient interface device of claim 1, wherein the at least one tightening indicator (48; 148) is a stick-like tightening element (50).

12. The patient interface device of claim 1, wherein the at least one tightening indicator (48; 148) is a chemical indicator for indicating the optimum tightening position by generating a chemically-induced signal caused by a face contact and/or a predetermined pressure.

13. The patient interface device of claim 1, wherein the patient interface (12; 112) comprises a first part (21; 121) and a second part (23; 123), wherein the first part (121) covers the mouth and/or the nose of the user and the second part is arranged at the user's forehead.

14. The patient interface device of claim 13, wherein the at least one tightening indicator (48; 148) is arranged at the first part (121).

15. A patient interface apparatus, comprising:
a patient interface device (10; 110) as claimed in claim 1 and
a tightening means (32; 132) being connected to the patient interface (12; 112) enabling the user to tighten the patient interface (12; 112) towards the user's face.
